(19) United States
(12) Patent Application Publication
(10) Pub. No.: US 2025/0205476 A1
(43) Pub. Date: Jun. 26, 2025

(54) CANNULA FOR BLOOD PUMP

(71) Applicant: ABIOMED, Inc., Danvers, MA (US)

(72) Inventors: Jairo Moux, Danvers, MA (US); Michael Finnegan, Danvers, MA (US); Cam Loughlin, Danvers, MA (US); Zhenghong Tao, Danvers, MA (US)

(73) Assignee: ABIOMED, Inc., Danvers, MA (US)

(21) Appl. No.: 18/987,735

(22) Filed: Dec. 19, 2024

Related U.S. Application Data
(60) Provisional application No. 63/614,093, filed on Dec. 22, 2023.

(51) Int. Cl. A61M 60/857; A61M 60/13; A61M 60/216; A61M 60/414

(52) U.S. Cl. CPC A61M 60/857; A61M 60/13; A61M 60/216; A61M 60/414; A61M 2205/3306

(57) ABSTRACT

A cannula for, e.g., a blood pump, may be provided. The cannula may include a tubular body having a first lumen extending from a first end to a second end. A coil may be disposed around the first lumen. The first lumen may be configured to allow blood to flow therethrough. The cannula may include optical fiber lumen extending along at least a portion of the tubular body. The cannula may include at least one optical fiber within each optical fiber lumen. The cannula may include support lumen. Each support lumen may extend along at least a portion of the tubular body. Each optical fiber lumen and each support lumen may be disposed within the tubular body. The cannula may include reinforcement member(s) within the support lumen. Each reinforcement member may be configured to provide a resistance to bending of the tubular body in at least one direction.

### CROSS-REFERENCE TO RELATED APPLICATIONS

The present application claims priority to U.S. Provisional Application No. 63/614,093 filed Dec. 22, 2023, which is incorporated by reference herein by its entirety.

### TECHNICAL FIELD

The present application is directed towards cannulas for blood pumps, and specifically, to cannulas with reinforcement members to resist bending in one or more directions.

### BACKGROUND

Modern blood pumps utilize sensors, and such sensors may include optical sensors. If a blood pump with a flexible cannula requires an optical sensor at a distal end, the optical sensor has to pass through the cannula before it can connect to the distal end. However, as is known, optical fibers are very brittle and fragile, and can fracture, rendering the sensor inoperable.

### BRIEF SUMMARY

In various aspects, a cannula may be provided. The cannula may include a tubular body (which may include, e.g., at least one thermoplastic polyurethane (TPU)), having a first end and a second end. The cannula may include a first lumen extending in an axial direction from the first end to the second end. A coil may be disposed around the first lumen. The coil may include, e.g., a metal or metal alloy. The first lumen may be configured to allow blood to flow therethrough. The tubular body may have a first effective outer diameter at the first end and a second effective outer diameter at the second end, where the first effective outer diameter is smaller than the second effective outer diameter.

The cannula may include one or more optical fiber lumen. Each optical fiber lumen may extend at least partially along at least a portion of the tubular body. Each optical fiber lumen may, independently, be disposed within the tubular body (i.e., either between the coil and the first lumen, or outside the coil). At least one optical fiber may be within each optical fiber lumen. The cannula may include one or more support lumen. Each support lumen may extend at least partially along a portion of the tubular body. Each support lumen may, independently, be disposed within the tubular body (i.e., between the coil and the first lumen, or outside the coil).

A number of the one or more optical fiber lumen may be less than a number of the one or more support lumen. A number of the one or more optical fiber lumen may be equal to a number of the one or more support lumen. A number of the one or more optical fiber lumen may be greater than a number of the one or more support lumen. The one or more optical fiber lumen may include a plurality of optical fiber lumen. Each optical fiber lumen may extend in an axial direction. At least one optical fiber lumen may include a curved portion that extends circumferentially and axially relative to a first end of the at least one of the one or more optical fiber lumen. At least one optical fiber lumen may have a length less than a length of the tubular body. At least one of the one or more optical fiber lumen may have a length no more than about ¼ the length of the tubular body. At least one of the one or more optical fiber lumen may have a length that is ¼ to ¾ the length of the tubular body. At least one of the one or more optical fiber lumen may have a length at least ¾ the length of the tubular body.

A number of the one or more support lumen may be an even number. A number of the one or more support lumen may be an odd number. Each optical fiber lumen may be disposed between the coil and an outer surface of the tubular body. Each support lumen may be disposed between the coil and an outer surface of the tubular body. At least one of the one or more support lumen may have a length less than a length of the tubular body. At least one of the one or more support lumen may have a length less no more than about ⅓ the length of the tubular body. At least one of the one or more support lumen may have a length ⅓ to ⅔ the length of the tubular body. At least one of the one or more support lumen may have a length at least ⅔ the length of the tubular body. At least one support lumen may have at least one tapered end.

The cannula may include at least one reinforcement member within each of the one or more support lumen. Each reinforcement member may be configured to have a stiffness greater than a stiffness of the at least one optical fiber. Each reinforcement member may be configured to provide a resistance to bending of the tubular body in at least one direction. Each reinforcement member may include, e.g., a metal, a metal alloy, or a polymer. Each reinforcement member may have a circular cross-section. Each reinforcement member may have a non-circular cross-section. Each reinforcement member may have a stiff portion and a flexible portion, wherein the flexible portion is located on at least one end of each reinforcement member.

The cannula may include at least one additional lumen extending at least partially along a portion of the tubular body. Each of the one or more additional lumen being disposed within the tubular body (i.e., either between the coil and the first lumen, or outside the coil). Each additional lumen may be disposed between the coil and an outer surface of the tubular body. One or more additional lumen may have a length less than a length of the tubular body. One or more of the at least one additional lumen may be configured to deliver a fluid through the one or more of the at least one additional lumen.

In various aspects, a blood pump may be provided. The blood pump may include a catheter. The blood pump may include a cannula as disclosed herein operably coupled to a distal end of the catheter. The blood pump may include a pump housing. The pump housing may be disposed between the catheter and the cannula. The first end of the cannula may be coupled to the pump housing.

### BRIEF DESCRIPTION OF DRAWINGS

The accompanying drawings, which are incorporated in and constitute a part of this specification, illustrate embodiments of the present invention and, together with a general description of the invention given above, and the detailed description of the embodiments given below, serve to explain the principles of the present invention.

FIG. 1 is an illustration of a system.

FIG. 2 is an illustration of a distal end of a blood pump.

FIG. 3A is an illustration of a cannula.

FIG. 3B-3K are illustrations of example cross-sections of a cannula of FIG. 3A.

FIG. 4A and 4B are illustrations of cannula with constant (4A) and varying (4B) diameters.

FIGS. 5A-5C are illustrations of example optical lumen in the cannula.

FIG. 5D is an illustration of a cross-section of the cannula shown in FIG. 5C.

FIGS. 6A-6B are illustrations of example support lumen in the cannula.

FIG. 7A is an illustration of a tapered end of a support lumen.

FIG. 7B is an illustration of a support lumen with varying diameters.

FIGS. 8A-8D are illustrations of reinforcement members.

FIG. 8E is an illustration of a simplified example cross-section of a cannula of FIG. 3A, showing only a support lumen with two reinforcement members (no other lumen shown for clarity).

FIG. 9 is an illustration of example additional lumen in the cannula.

It should be understood that the appended drawings are not necessarily to scale, presenting a somewhat simplified representation of various features illustrative of the basic principles of the invention. The specific design features of the sequence of operations as disclosed herein, including, for example, specific dimensions, orientations, locations, and shapes of various illustrated components, will be determined in part by the particular intended application and use environment. Certain features of the illustrated embodiments have been enlarged or distorted relative to others to facilitate visualization and clear understanding. In particular, thin features may be thickened, for example, for clarity or illustration.

### DETAILED DESCRIPTION

The following description and drawings merely illustrate the principles of the invention. It will thus be appreciated that those skilled in the art will be able to devise various arrangements that, although not explicitly described or shown herein, embody the principles of the invention and are included within its scope. Furthermore, all examples recited herein are principally intended expressly to be only for illustrative purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or, unless otherwise indicated (e.g., “or else” or “or in the alternative”). Also, the various embodiments described herein are not necessarily mutually exclusive, as some embodiments can be combined with one or more other embodiments to form new embodiments.

The numerous innovative teachings of the present application will be described with particular reference to the presently preferred exemplary embodiments. However, it should be understood that this class of embodiments provides only a few examples of the many advantageous uses of the innovative teachings herein. In general, statements made in the specification of the present application do not necessarily limit any of the various claimed inventions. Moreover, some statements may apply to some inventive features but not to others. Those skilled in the art and informed by the teachings herein will realize that the invention is also applicable to various other technical areas or embodiments.

As it is known, percutaneous blood pumps may be used to support a patient's heart. Such blood pumps may be inserted percutaneously and pass through the patient's vasculature. As it is also known, such blood pumps may include an inlet, a cannula, an outlet, an impeller, a catheter and a motor. Blood pumps may also include optical sensors, and thus may include optical fibers, which are enclosed in a lumen extending along the length of the catheter and/or at least a portion of the length of the cannula. However, as it is also known, while inserting the percutaneous blood pump, the cannula may kink due to the patient's vasculature. Accordingly, the optical fiber and/or the optical fiber lumen may be vulnerable to kinking as the cannula kinks. In some instances, the optical fiber may be damaged and/or inoperable after kinking.

Thus, the inventors recognize and appreciate the benefit of including supporting lumens on the cannula to minimize and/or prevent kinking of the optical fiber lumen. The supporting lumens may be located in desired locations to minimize and/or prevent the optical fiber lumen from kinking and, in turn, minimize and/or prevent the optical fiber from being damaged during insertion of the blood pump. The inventors have also recognized the benefit of including a reinforcement member inside of the support lumen.

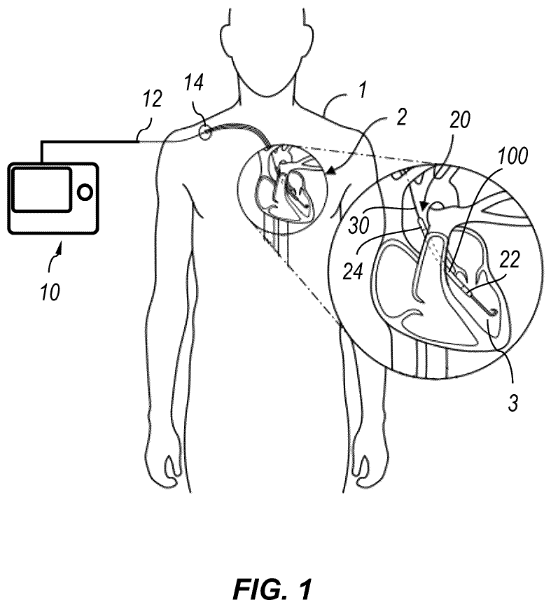

In various aspects, a blood pump may be provided. Referring to FIG. 1, a blood pump 20 may be deployed within a blood vessel of a patient 1, such as in a heart 2. The blood pump may include a cannula 100 operably coupled to a distal end of a catheter 30. The blood pump may be configured to cause blood to flow into an inlet 22, through the cannula 100, and out of a blood-flow outlet 24. Here, the blood flow inlet is shown as being disposed within a left ventricle 3, but as will be understood, the pump could be deployed at other locations within a patient. For example, the blood flow inlet may be disposed within a right ventricle. The pump may be operably coupled to a controller 10 via, e.g., one or more wires 12 or tubes through an access point 14.

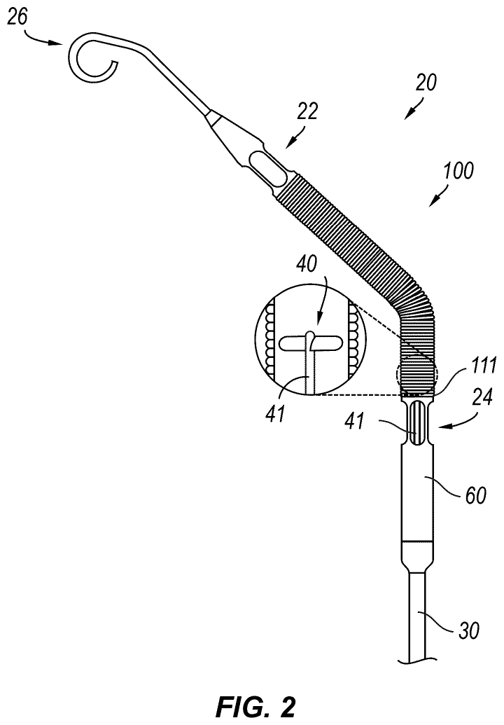

For example, referring to FIG. 2, a blood pump 20 can be seen, having a cannula 100 coupled to a distal end of catheter 30. A flexible atraumatic tip 26 may be coupled to a distal end of the blood pump. The cannula 100 may, optionally, include an impeller 40 disposed within the cannula 100. Optionally, the impeller may be within a housing 60. Housing 60 may include, e.g., the impeller and/or an electric motor. The housing (which may sometimes be referred to as a pump housing) may be disposed between the catheter and the cannula. In some embodiments, a first end 111 of the cannula may be coupled to the pump housing.

The motor may be coupled to the impeller via a drive shaft 41, which may be a rigid drive shaft. In some embodiments, when the impeller rotates, blood is configured to flow through an inlet 22, through the cannula, and out an outlet 24. In some embodiments, the motor may be located outside of the patient. In such embodiments, the drive shaft 41 may be a flexible driveshaft.

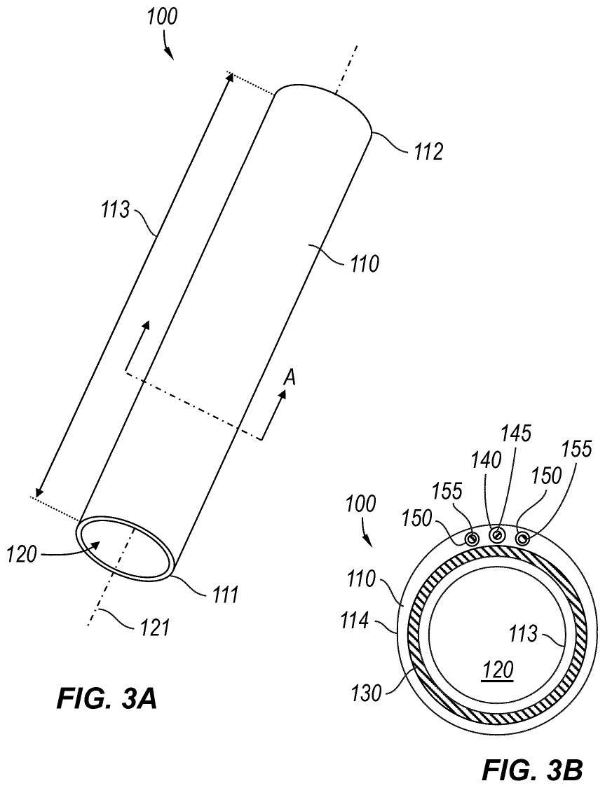

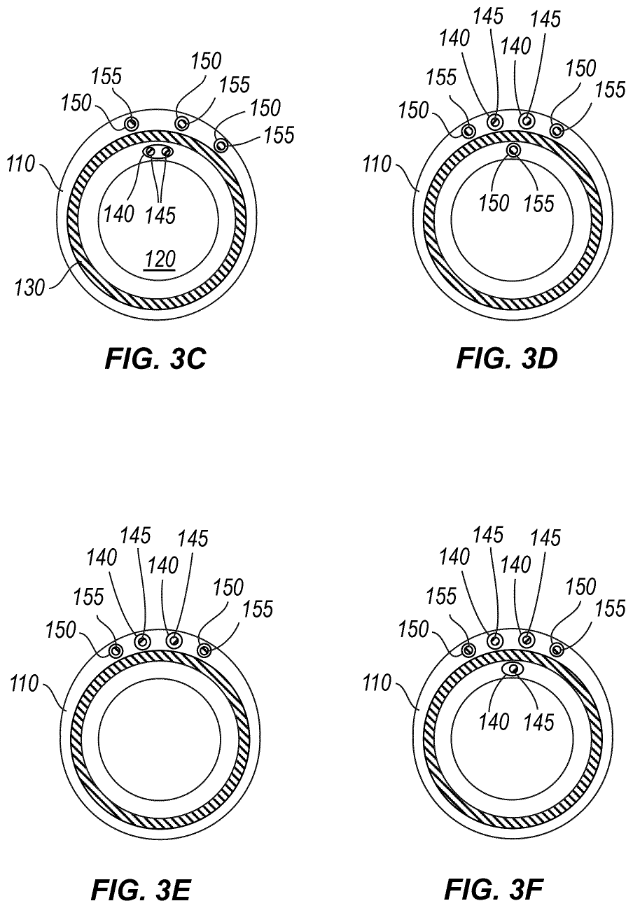

In various aspects, a cannula may be provided. Referring to FIG. 3A, the cannula 100 may include a tubular body 110 having a first end 111 and a second end 112. The tubular body may be composed of any appropriate material, such as at least one thermoplastic polyurethane (TPU). The cannula may have a length 113.

The cannula may include a first lumen 120 extending in an axial direction 121 from the first end to the second end. Referring to FIG. 3B, the tubular body may include an inner surface 118 that defines the first lumen. The tubular body may include an outer surface 114. The first lumen may be configured to allow blood to flow therethrough.

The cannula may include a coil or tubular member 130. In some aspects, the coil (e.g., coil or tubular member 130) may be disposed around the first lumen, e.g., within the tubular body, i.e., between the inner surface 118 and outer surface 114. In some embodiments, the coil may be disposed around the tubular body. The coil may include, e.g., a metal or metal alloy.

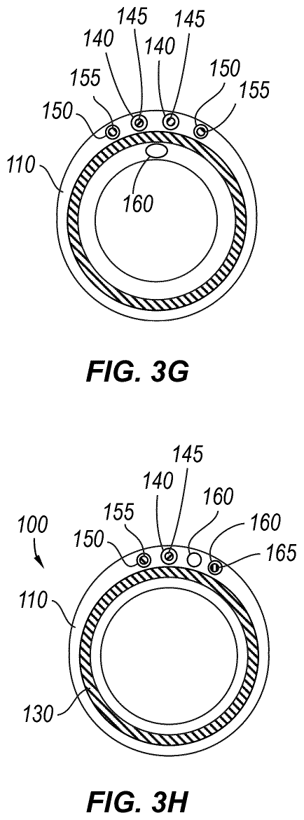

Referring to FIG. 4A and 4B, the tubular body may have a substantially constant effective diameter 401 along the length of the tubular body. As used herein, “substantially constant” is intended to refer to a dimension that varies less than 10%, such as less than 5%, or even less than 2%. As seen in FIG. 4B, the tubular body may have a first effective outer diameter 402 at the first end 111 and a second effective outer diameter 403 at the second end 112, where the first effective outer diameter is smaller than the second effective outer diameter.

Referring to FIG. 3B, the cannula may include one or more optical fiber lumen 140. Each optical fiber lumen may, independently, be disposed within the tubular body (i.e., either between the coil and the first lumen, or outside the coil). In FIG. 3B, the optical fiber lumen is shown outside the coil. In FIG. 3C, the optical fiber lumen is shown between the coil or tubular member 130 and the first lumen 120. As shown in FIGS. 3B and 3C, at least one optical fiber 145 may be within each optical fiber lumen 140. In some embodiments (see 3B), a single optical fiber may be within each optical fiber lumen. In some embodiments (see 3C), a plurality of optical fibers may be within each optical fiber lumen. In FIGS. 3B and 3C, the cannula is shown as having a single optical fiber lumen. However, referring to FIG. 3D, the cannula may include a plurality of optical fiber lumen. Each may, independently, be disposed either between the coil and the first lumen, or outside the coil. As will be appreciated, the optical fiber lumen may be made of any suitable material and/or any suitable stiffness.

In some embodiments, the cannula may include one or more lumen. In such embodiments, each of the one or more lumen may have the same properties and features described above with respect to the optical fiber lumen 140 of FIG. 4B. However, in some aspects, one of the one or more lumen may be configured for an optical fiber to pass through (e.g., to slidably receive an optical fiber) and one of the one or more lumen may be configured to receive a non-optical cable or fiber. The non-optical cable or fiber may be, e.g., an electrical cable. For example, one or more sensor cables may pass through the one or more lumen.

In other embodiments, coil or tubular member 130 may be a tubular member. The tubular member 130 may extend along at least a portion of the length of the tubular body 110. For example, the tubular member 130 may extend along the entire length of the tubular body 110. In other embodiments, the tubular member 130 may extend along only a portion of the tubular body 110. As will be appreciated, the tubular member 130 may be of any suitable geometry. For example, the tubular member 130 may have a circular cross-sectional shape. The tubular member 130 may have a variable geometry along the length of the tubular body 110. For example, the tubular member 130 may have a first diameter at or near the first end 111 and a second diameter at or near the second end 112.

In some embodiments, the one or more optical fiber lumen may have an inner diameter of about 400 micron to about 500 micron. As will be appreciated, the one or more optical fiber lumen may have any suitable inner diameter. In other embodiments, each of the one or more optical fiber lumen may have different inner diameters. While diameter is used, there is no requirement that the one or more optical fiber lumen have a circular cross-section, as will be discussed herein. In some embodiments, diameter may refer to the largest cross-sectional measurement. For example, in some embodiments, the one or more optical fiber lumen may have a quadrilateral shape (as will be discussed herein) and thus the diameter may refer to the largest cross-sectional measurement.

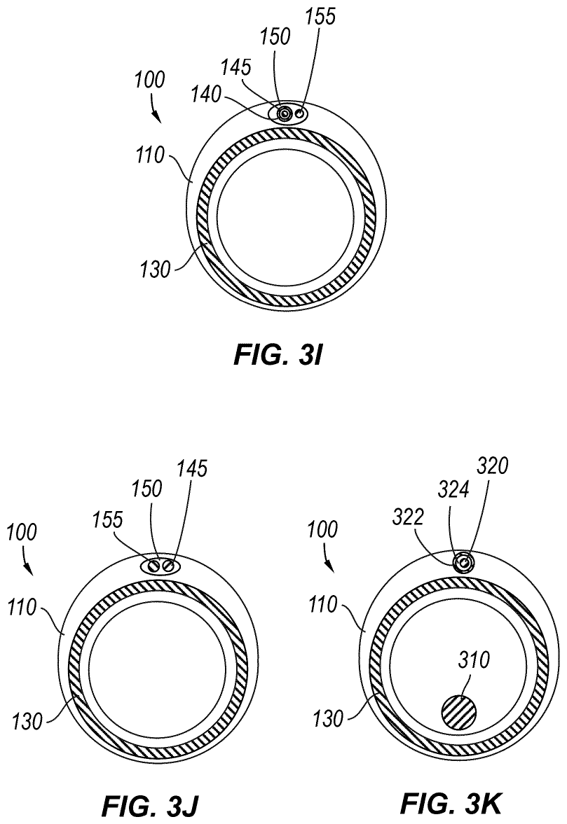

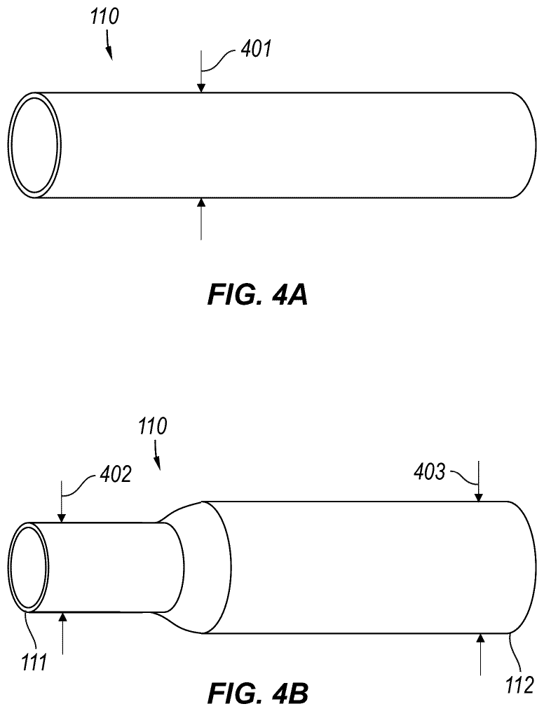

Referring to FIG. 5A, each optical fiber lumen may extend at least partially along at least a portion 115 of the tubular body. As shown, each optical fiber lumen 140 may extend from a first end 111 towards a second end 112. Each optical fiber lumen may extend a length 501 within the tubular body. Referring to FIG. 5B, the optical fiber lumen 140 may begin a distance 502 from the first end 111, where the distance 502 is greater than 0. That is, a portion 510 of an optical fiber may be outside the outer surface 114 of the tubular body, and may pass into the optical fiber lumen at a distance 502 from the first end. The optical fiber lumen 140 may end a distance 503 from the second end 112, where the distance 503 is greater than 0. In some embodiments, at least one optical fiber lumen may extend the entire length of the tubular body.

At least one optical fiber lumen may have a length 501 less than a length 113 of the tubular body. At least one of the one or more optical fiber lumen may have a length no more than about ¼ the length of the tubular body. At least one of the one or more optical fiber lumen may have a length that is ¼ to ¾ the length of the tubular body. At least one of the one or more optical fiber lumen may have a length at least ¾ the length of the tubular body.

Referring to FIGS. 5A and 5B, each optical fiber lumen may extend in an axial direction 121. Referring to FIGS. 5C and 5D, at least one optical fiber lumen 140 may include a curved portion 520 that extends circumferentially 521 and axially 121 relative to a first end 141 of the optical fiber lumen 140. The optical fiber lumen may extend from the first end 141 to a second end 142. In some embodiments, the angle 522 of rotation around a central axis 523 of the tubular body 110 formed by a curved portion 520 of the optical fiber lumen (e.g., when viewed axially as in FIG. 5D, from a starting point 524 of the curved portion to an end point 525 of the curved portion) is less than 360°. In some embodiments, the angle is no more than 270°. In some embodiments, the angle is no more than 180°. In some embodiments, the angle is no more than 90°. In some embodiments, the angle is at least 45°. In some embodiments, the angle is at least 90°.

Referring to FIGS. 3B-3D, the cannula may include one or more support lumen 150. Each support lumen may, independently, be disposed within the tubular body (i.e., between the coil and the first lumen, or outside the coil). As will be appreciated, the support lumen may be made of any suitable material. For example, the support lumen may be made of a material that is stiffer than the material of the optical fiber lumen.

As will be understood, the number of optical fiber lumen may vary. The number of optical fiber lumen may be less than a number of support lumen (see, e.g., FIGS. 3B-3D). The number of optical fiber lumen may be equal to the number of support lumen (see, e.g., FIG. 3E). The number of optical fiber lumen may be greater than the number of support lumen (see, e.g., FIG. 3F).

The number of support lumen may be an even number (see, e.g., FIG. 3E). The number of support lumen may be an odd number (see, e.g., FIGS. 3C, 3D). Each optical fiber lumen may be disposed between the coil and an outer surface of the tubular body (see, e.g., FIGS. 3B, 3D). Each support lumen may be disposed between the coil and an outer surface of the tubular body (see, e.g., FIGS. 3B-3C, 3E-3F).

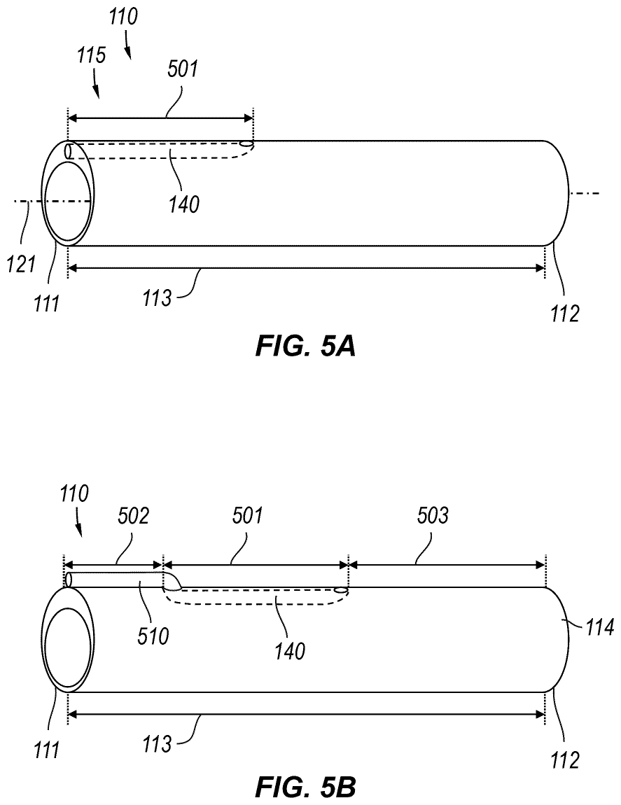

Referring to FIG. 6A, each support lumen may extend at least partially along at least a portion 116 of the tubular body. As shown, each support lumen 150 may extend from a first end 111 towards a second end 112. Each support lumen may extend a length 601 within the tubular body. Each support lumen may have a diameter 154. As shown in FIG. 6A, the diameter 154 of the support lumen may be constant. Referring to FIG. 6B, the support lumen 150 may begin a distance 602 from the first end 111, where the distance 602 is greater than 0. The support lumen 150 may end a distance 603 from the second end 112, where the distance 603 is greater than 0.

At least one support lumen may have a length 601 less than a length 113 of the tubular body. At least one of the one or more support lumen may have a length 601 of no more than about ¼ the length of the tubular body. At least one of the one or more support lumen may have a length that is ¼ to ¾ the length of the tubular body. At least one of the one or more support lumen may have a length at least ¾ the length of the tubular body.

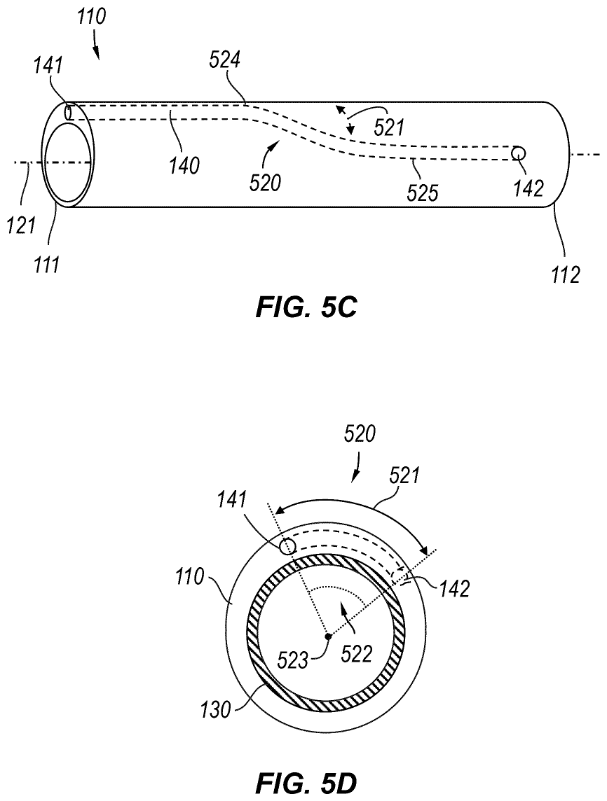

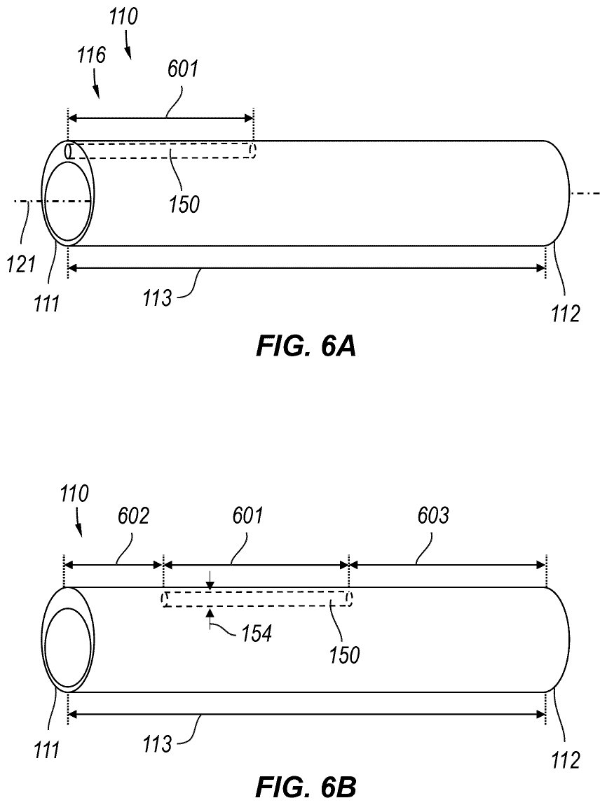

Referring to FIG. 7A, at least one support lumen 150 may have at least one tapered end 720. In some embodiments, one or more support lumen may have both ends tapered. In some embodiments, all support lumen have at least one tapered end.

Referring to FIG. 7B, the effective diameter of the support lumen may vary in an axial direction. For example, a support lumen 150 may have a first portion 701 with a first effective diameter 711, that may change to a second effective diameter 712 at a second portion 702 that is, e.g., more distal to the first portion. As shown, a third portion 703 may have a third effective diameter 713, and a fourth portion 704 may have a fourth effective diameter 714. In this manner, additional control over the flexibility of the cannula can be achieved.

Referring to FIG. 3B, the cannula may include at least one reinforcement member 155 within each of the one or more support lumen 150. Each reinforcement member may be configured to have a stiffness greater than a stiffness of the at least one optical fiber. Each reinforcement member may be configured to provide a resistance to bending of the tubular body in at least one direction. Each reinforcement member may include, e.g., a metal, a metal alloy, or a polymer.

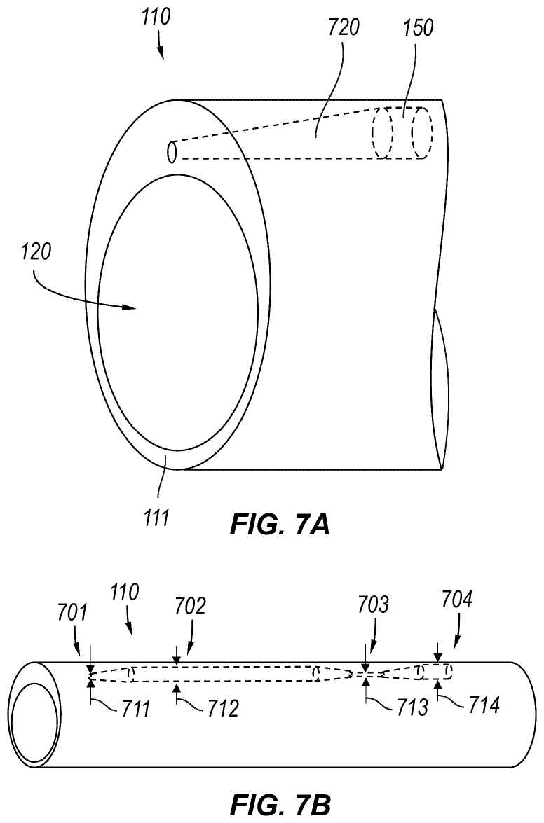

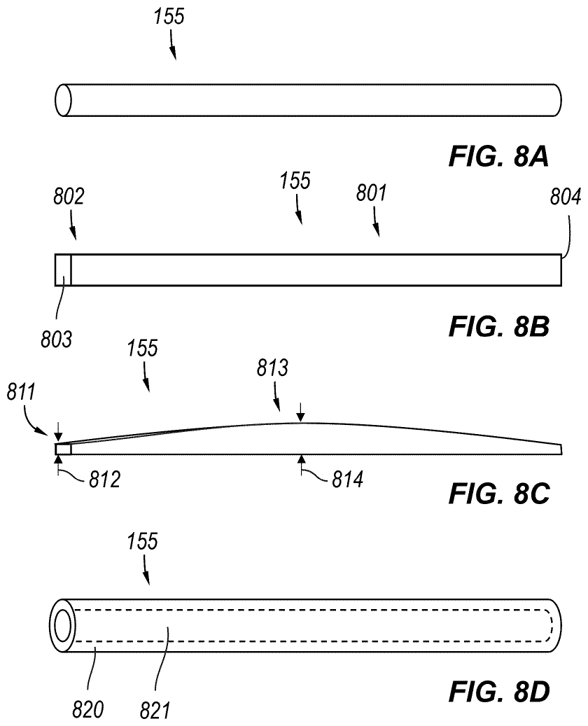

Referring to FIG. 8A, each reinforcement member may have a circular cross-section. Referring to FIG. 8B, each reinforcement member may have a non-circular cross-section. Each reinforcement member may have a stiff portion 801 and a flexible portion 802, wherein the flexible portion is located on at least one end 803, 804 of each reinforcement member. In some embodiments, the stiff portion 801 and/or the flexible portion 802 may be located on the cannula in order to achieve desired shape retention properties for the cannula. For example, one or more stiff portions 801 may be located in positions on the cannula in order to maintain a desired geometry during insertion and/or operation of the blood pump. As will be appreciated, any suitable number of stiff portions 801 and/or flexible portions 802 may be used. As will be further appreciated, the number of stiff portions and flexible portions may be the same or may vary.

Each reinforcement member may have a substantially constant thickness or diameter. (see, e.g., FIGS. 8A, 8B). Each reinforcement member may have a varying thickness or diameter (see, e.g., FIG. 8C). For example, each reinforcement member may have a first portion 811 having a first thickness or effective diameter 812, and second portion 813 with a different thickness or effective diameter 814.

Referring to FIG. 8D, in some embodiments, a reinforcement member may have an outer layer 820. The outer layer may be disposed around an inner layer 821, or may be disposed around air (e.g., forming a hollowing reinforcement member). The outer layer and inner layer may be of different materials.

Referring to FIG. 8E, in some embodiments, two or more reinforcement members may be present within a single support lumen. As shown, a first reinforcement member 920 is disposed alongside a second reinforcement member 921 within support lumen 150. For clarity, no other lumen are shown. In FIG. 8E, a side-by-side arrangement is shown. However, other arrangements may be utilized. In some embodiments, the two reinforcement members may be braided. In some embodiments, the two reinforcement members may be layered.

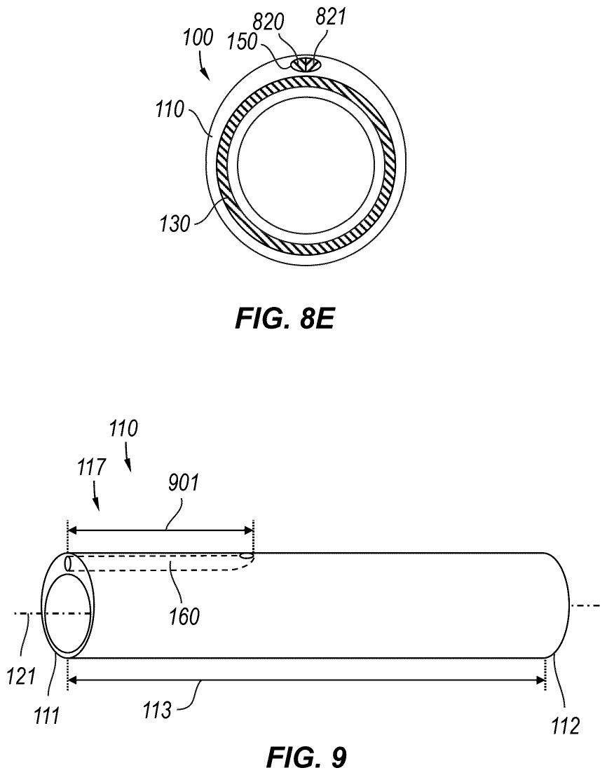

Referring to FIGS. 3G and 3H, the cannula may include at least one additional lumen 160. Each of the one or more additional lumen being disposed within the tubular body (i.e., either between the coil and the first lumen, or outside the coil). One or more of the at least one additional lumen may be configured to deliver a fluid through the one or more of the at least one additional lumen. One or more electrical wires 165 may be disposed within at least one additional lumen 160. Each additional lumen may be disposed between the coil and an outer surface of the tubular body (see FIG. 3H). Referring to FIG. 9, each additional lumen may extend at least partially along a portion 117 of the tubular body. One or more additional lumen may have a length 901 less than a length 113 of the tubular body.

Referring to FIGS. 3I and 3J, in some aspects, the optical fiber and/or optical fiber lumen may be located within a support lumen. For example, as shown in FIG. 3I, in some aspects, an optical fiber 145 may be disposed with an optical fiber lumen 140, that is disposed within a support lumen 150 that also contains a reinforcement member 155. In some aspects, an optical fiber 145 and/or a reinforcement member may be disposed with a support lumen 150. In such embodiments shown in FIGS. 3I and 3J, electrical cables may be disposed within the first lumen 120. As will be appreciated, any suitable electrical cables may be disposed within the first lumen. For example, motor cables may be disposed within the first lumen. As will also be appreciated, any suitable type of sensor cable may be disposed within the optical fiber lumen 140.

Referring to FIG. 3K, in some aspects, one or more cables or fibers 310 may be disposed within coil or tubular member 130. Such cables or fibers may be any appropriate type of cable or fiber (e.g., an electrical cable, an optical fiber, etc.).

In some aspects, one or more cables or fibers 320 may be disposed within support lumen 150, such as within a lumen 324 defined by a coil or reinforcement member 322. Such cables or fibers may be any appropriate type of cable or fiber (e.g., an electrical cable, an optical fiber, etc.).

Various modifications may be made to the systems, methods, apparatus, mechanisms, techniques, and portions thereof described herein with respect to the various figures, such modifications being contemplated as being within the scope of the invention. For example, while a specific order of steps or arrangement of functional elements is presented in the various embodiments described herein, various other orders/arrangements of steps or functional elements may be utilized within the context of the various embodiments. Further, while modifications to embodiments may be discussed individually, various embodiments may use multiple modifications contemporaneously or in sequence, compound modifications and the like.

Although various embodiments which incorporate the teachings of the present invention have been shown and described in detail herein, those skilled in the art can readily devise many other varied embodiments that still incorporate these teachings. Thus, while the foregoing is directed to various embodiments of the present invention, other and further embodiments of the invention may be devised without departing from the basic scope thereof. As such, the appropriate scope of the invention is to be determined according to the claims.

1. A cannula, comprising:
a tubular body having a first end, a second end, a first lumen extending in an axial direction from the first end to the second end, wherein the first lumen is configured to allow blood to flow therethrough;
a coil disposed around the first lumen;
one or more optical fiber lumen extending at least partially along at least a portion of the tubular body, each of the one or more optical fiber lumen is disposed within the tubular body, either between the coil and the first lumen or outside the coil;
at least one optical fiber within each optical fiber lumen;
one or more support lumen, each of the one or more support lumen extending at least partially along a portion of the tubular body, each of the one or more support lumen being disposed within the tubular body, either between the coil and the first lumen or outside the coil; and
at least one reinforcement member within each of the one or more support lumen, each reinforcement member configured to have a stiffness greater than a stiffness of the at least one optical fiber, each reinforcement member configured to provide a resistance to bending of the tubular body in at least one direction.

2. The cannula of claim 1, wherein a number of the one or more optical fiber lumen is less than a number of the one or more support lumen.

3. The cannula of claim 1, wherein a number of the one or more optical fiber lumen is equal to a number of the one or more support lumen.

4. The cannula of claim 1, wherein a number of the one or more optical fiber lumen is greater than a number of the one or more support lumen.

5. The cannula of claim 1, wherein the one or more optical fiber lumen comprises a plurality of optical fiber lumen.

6-7. (canceled)

8. The cannula of claim 1, wherein each of the one or more optical fiber lumen are disposed between the coil and an outer surface of the tubular body.

9. (canceled)

10. The cannula of claim 1, wherein each of the one or more optical fiber lumen extend in an axial direction.

11. The cannula of claim 1, wherein at least one of the one or more optical fiber lumen includes a curved portion that extends circumferentially and axially relative to a first end of the at least one of the one or more optical fiber lumen.

12. The cannula of claim 1, wherein at least one of the one or more optical fiber lumen has a length less than a length of the tubular body.

13-15. (canceled)

16. The cannula of claim 1, wherein at least one of the one or more support lumen has a length less than a length of the tubular body.

17-19. (canceled)

20. The cannula of claim 1, wherein the tubular body has a first effective outer diameter at the first end and a second effective outer diameter at the second end, the first effective outer diameter being smaller than the second effective outer diameter.

21. The cannula of claim 1, further comprising at least one additional lumen extending at least partially along a portion of the tubular body, each of the one or more additional lumen being disposed within the tubular body, either between the coil and the first lumen or outside the coil.

22-24. (canceled)

25. The cannula of claim 1, wherein the tubular body is comprised of at least one thermoplastic polyurethane (TPU).

26. The cannula of claim 1, wherein the coil is comprised of a metal or metal alloy.

27. The cannula of claim 1, wherein each reinforcement member is comprised of a metal, a metal alloy, or a polymer.

28-29. (canceled)

30. The cannula of claim 1, wherein at least one of the one or more support lumen has at least one tapered end.

31. The cannula of claim 1, wherein each reinforcement member has a stiff portion and a flexible portion, wherein the flexible portion is located on at least one end of each reinforcement member.

32. A blood pump, comprising:
a catheter; and
a cannula of claim 1 operably coupled to a distal end of the catheter.

33. The blood pump of claim 32, further comprising a pump housing, the pump housing being disposed between the catheter and the cannula.

34. The blood pump of claim 33, wherein the first end of the cannula is coupled to the pump housing.
